(19) United States
(12) Patent Application Publication
(10) Pub. No.: US 2017/0312122 A1
(43) Pub. Date: Nov. 2, 2017

(54) HYPERTHERMIA THERAPY MASK

(71) Applicant: THE FIRST AFFILIATED HOSPITAL OF GUANGZHOU MEDICAL UNIVERSITY, Guangzhou (CN)

(72) Inventors: Xiaowen ZHANG, Guangzhou (CN); Xiaoming FANG, Guangzhou (CN); Lijuan SONG, Guangzhou (CN); Zhengguo ZHANG, Guangzhou (CN); Xiaolong ZHAO, Guangzhou (CN)

(21) Appl. No.: 15/655,837

(22) Filed: Jul. 20, 2017

Related U.S. Application Data
(63) Continuation of application No. PCT/CN2015/076446, filed on Apr. 13, 2015.

(30) Foreign Application Priority Data
Jan. 27, 2015 (CN) 201510042220.5

(51) Int. Cl. A61F 7/02; A61F 7/00

(52) U.S. Cl. CPC A61F 7/02; A61F 2007/0292; A61F 2007/0006; A61F 2007/0228

(57) ABSTRACT

The present invention relates to a hyperthermia mask used in the field of medical appliances. The hyperthermia mask of the present invention comprises a mask surface body and a tensioning belt, the mask surface body is made of phase change fibers or the mask surface body is filled with the phase change fibers; the phase change fibers are composed of hollow fibers and phase change materials filled into the hollow fibers; and the phase change materials have a phase change temperature of 40° C.-55° C. The hyperthermia mask of the present invention is prepared based on phase change heat storage, is convenient to use and has a good hyperthermia effect.

### TECHNICAL FIELD

The present invention relates to the field of medical appliances, and more specifically, to a hyperthermia mask.

### BACKGROUND OF THE INVENTION

Inflammatory diseases of a nasal cavity and its adjacent tissues, such as chronic sinusitis, allergic rhinitis and acute rhinitis, are global health problems having high incidence, great harm to patients and society and large economic burden in human diseases. Antibiotics, hormones, antihistamines and other drugs are common means of treating these diseases, but these drugs have great side effects after being used for a long term.

Researches show that cold air stimulation is a factor of aggravating and relapsing chronic airway inflammation. Recently, increasing evidence shows that local hyperthermia can significantly improve nasal symptoms. Hyperthermia is a treatment means for heating tissues and organs or the whole body by a physical method. At present, clinical applications of the hyperthermia are classified into three categories: local hyperthermia (comprising superficial heating, cavity hyperthermia and implantation hyperthermia), regional hyperthermia (comprising deep tumor heating and various hyperthermal perfusion techniques) and whole-body hyperthermia. Warming has effects of stimulating antigen-presenting cells, promoting tissue cells to express heat shock proteins, and activating innate immunity. The hyperthermia enables local vasodilatation, increases a blood flow speed to be conducive to expelling toxins in the tissues, also promotes blood circulation, increases a blood flow volume, speeds up metabolism, and enhances a phagocytic function of white blood cells. Thus, in an early stage of inflammation, the heat can be used to promote absorption and dissipation of inflammatory exudates; and in a later stage of the inflammation, the heat can be used to help removal of necrotic tissues and repair of the tissues because leukocytes release proteolytic enzyme to dissolve the necrotic tissues, so that the inflammation is limited. Common hyperthermia methods comprise microwave, radio frequency, ultrasound, magnetic liquid heating, infrared ultrasound, water bath, air bath, etc.

20 years ago, some scholars had studied and confirmed that intranasal inhalation of hot steam (41° C. and 43° C.) can improve symptoms of rhinitis and reduce contents of nasal local histamine and leukotriene 4 (see Georgitis J W. Local hyperthermia and nasal irrigation for perennial allergic rhinitis: effect off symptoms and nasal airflow. Ann Allergy. 1993; 71: 385-389, and Georgitis J W. Nasal hyperthermia and simple irrigation for perennialrhinitis. Changes in inflammatory mediators. Chest. 1994; 106(5): 1487-1492.). A prospective randomized double-blind study shows that for 70 patients with chronic rhinitis after treatment by nasal irrigation with hot water for 1 month, a nasal endoscopy score, a nasal resistance, the number of nasal cilia are significantly better than those in treatment with an isotonic sodium chloride solution (see Ottaviano G, Marioni G, Giacomelli L, La Torre F B, Staffieri C, Marchese-Ragona R, Staffieri A. Smoking and chronic rhinitis: effects of nasal irrigations with sulfurous-arsenical-ferruginous thermal water: A prospective, randomized, double-blind study. Am J Otolaryngol. 2012; 33 (6): 657-62.). Satisfactory effects are also achieved in application of SPA and traditional Chinese medicines for fumigation treatment of chronic sinusitis in China and abroad. It is reported that (see Garavello W, Romagnoli M, Sordo L, et al. Hypersaline nasal irrigation in children with symptomatic seasonal allergic rhinitis: a randomized study. Pediatr Allergy Immunol, 2003.14 (2): 140-3.) nasal irrigation fluid prepared from hypertonic saline at a room temperature (25° C.) can be used for nasal irrigation treatment of seasonal AR to achieve better efficacy. Domestic scholars study (see Lin Lin, Yan Wenhong and Zhao Xia. Treatment Effect of Saline Nasal Irrigation at Different Temperatures for Allergic Rhinitis. Chinese Journal of Otorhinolaryngology Head and Neck Surgery, 2014; 49: 109-114.) and confirm that saline nasal irrigation at 40° C. can apparently improve sneezing and nasal congestion symptoms of AR patients and can apparently reduce the contents of nasal mucosal histamine and leukotriene 4. Rabago et al. believe that the nasal irrigation can reduce nasal mucosal edema, promote swinging of nasal mucosal epithelial cilia, clean up allergens attached to a nasal mucosal surface, reduce the content of nasal local inflammatory reaction factors, and also reduce asthma attack and prevent the incidence of nasal polyps after such a treatment mode is used for a long term (see Rabago D I, Guerard E, Bukstein D. Nasal irrigation for chronic sinus symptoms in patients with allergic rhinitis, asthma, and nasal polyposis: a hypothesis shooting study. WJJ. 2008 April; 107(2): 69-75.). These studies indicate that inhalation of gas or liquid at 43° C. can safely and effectively treat the nasal inflammation, but such a method has defects that the acting time is short, the gas or liquid is greatly affected by environments and is hard to keep constant, etc., and the water bath and the air bath require a special device, which is unaffordable to ordinary people.

Far-infrared has strong penetration force and radiation force, has a significant temperature control effect and resonance effect, and is easy to be absorbed by objects and converted into an internal energy of the objects. The far-infrared absorbed by a human body can make water molecules in the human body resonate, activate water molecules, and enhance the intermolecular binding force, thereby activating protein and other biological macromolecules, and causing that biological cells are at a highest vibration level. Since the biological cells generate the resonance effect, the far-infrared heat energy can be transferred to subcutaneous relatively-deep parts of the human body, the temperature of the deep layer rises, and the generated warm is diffused from inside to outside. Such action intensity enables telangiectasia, promotes the blood circulation, strengthens the metabolism between various tissues, increases regeneration ability of the tissues, improves the immunity of the body, and regulates abnormal excitement state of spirit, to play a role of health care. Hu et al. use a WS TY101 far-infrared emitter to face nose of a patient at a distance of 30 cm for 40 minutes every time for 7 days. During far-infrared treatment, eye itching, nasal itching, nasal congestion, nasal mucus, sneezing and other symptoms are significantly improved; dysosmia is not improved until the last treatment; and no apparent adverse effect appears (see Hu K H, Li W T. Clinical effects of far-infrared therapy in patients with allergic rhinitis. Conf Proc IEEE Eng Med Biol Soc. 2007; 2007: 1479-82.). Human body tissues have relatively strong absorption power for ultrasonic energy, so when the ultrasound is spread in the human body tissues, the energy is constantly absorbed by the tissues and becomes heat; as a result, the temperature of the tissues is increased. A heat production process is an energy conversion process of converting mechanical energy into the heat energy in a medium, i.e., endogenous heat. An ultrasonic warming effect can increase the blood circulation, speed up the metabolism, improve nutrition of local tissues, and enhance activity of the enzyme. Generally, the thermal effect of ultrasound is significantly reflected in bone and connective tissues, and minimally reflected in lipid and blood. Low-frequency ultrasound and low-frequency pulsed ultrasound can destroy a bacterial biofilm and also treat the chronic sinusitis. However, due to the high price of the device, it is difficult to meet needs of a large number of patients with rhinitis. Therefore, our research direction is to look for a hyperthermia product with low price and use convenience.

In summary, the existing technology for treatment of rhinitis with hyperthermia requires a special device, will consume a lot of time and money of the patients during the treatment of rhinitis, and is inconvenient.

### SUMMARY OF THE INVENTION

The purpose of the present invention is to overcome the above defects of the prior art to provide a hyperthermia mask which is based on phase change heat-storage and is convenient for treatment of nasal inflammation.

In order to achieve the above purpose, the present invention adopts a technical solution that the hyperthermia mask comprises a mask surface body and a tensioning belt, wherein the mask surface body is made of phase change fibers or the mask surface body is filled with the phase change fibers; the phase change fibers are composed of hollow fibers and phase change materials filled into the hollow fibers; and the phase change materials have a phase change temperature of 40° C.-55° C.

The phase change materials may store or release energy during phase change, and have a temperature substantially kept unchanged. When the phase change materials with the phase change temperature of 40° C.-55° C. are used as fillers of the hollow fibers and the hyperthermia mask is worn, as long as the phase change materials of the mask is subjected to phase change, the temperature of the mask can be ensured in a scope of 40° C.-55° C. Existing studies show that if the temperature of the mask is too high, the mask will hurt skin and damage blood vessels; and if the temperature of the mask is too low, the mask cannot play a role of hyperthermia.

As a preferred embodiment of the hyperthermia mask of the present invention, the phase change materials are filled into the hollow fibers by an adsorption method, a blending method or a dipping method.

As a preferred embodiment of the hyperthermia mask of the present invention, the phase change material is paraffin. The paraffin as an organic phase change heat storage material has advantages of high latent heat of phase change, adjustable phase change temperature, stable performance, no poison, etc.

As a more preferred embodiment of the hyperthermia mask of the present invention, the filling rate of the paraffin in the hollow fibers is 80%-90%. When the filling rate of the paraffin in the hollow fibers is greater than 80%, the latent heat of the phase change fibers is sufficiently high, and the hyperthermia time of the hyperthermia mask made of the phase change fibers or filled with the phase change fibers can reach more than 30 minutes, so the mask has a better hyperthermia effect. Meanwhile, the filling rate of the paraffin in the hollow fibers is less than 90%, which can prevent the paraffin from melting and leaking.

As a preferred embodiment of the hyperthermia mask of the present invention, the phase change fibers are prepared by a method comprising the following steps: firstly, heating the paraffin to a molten state; then, immersing one end of each hollow fiber in the paraffin, inserting the other end of each hollow fiber into one end of a rubber tube, and sealing a connection port of the hollow fibers and the rubber tube; and finally, connecting the other end of the rubber tube with a vacuum pump to suck the paraffin into the hollow fibers.

As a preferred embodiment of the hyperthermia mask of the present invention, an opening with a zipper is further formed in the mask surface body; and the phase change fibers are filled into the mask surface body through the opening with the zipper. The opening is formed in the mask surface body so that the phase change fibers are conveniently filled into the mask surface body and the phase change fibers are replaced conveniently. The zipper is arranged on the opening to facilitate the opening or closing of the opening in the mask surface body; and the opening is closed to be beneficial for heat preservation of the hyperthermia mask.

As a more preferred embodiment of the hyperthermia mask of the present invention, textiles are arranged on upper surfaces and lower surfaces of the phase change fibers. More preferably, the textiles are gauzes or cotton cloth. The textiles are arranged on the upper surfaces and the lower surfaces of the phase change fibers, so that the phase change fibers can be more conveniently placed in the mask surface body, and the mask has a better heat preservation effect.

A method for preparing the hyperthermia mask of the present invention comprises the following steps: (1) filling the phase change materials into micropores of the hollow fibers to prepare the phase change fibers; and (2) spinning the phase change fibers into the mask or filling the phase change fibers into the mask to obtain the hyperthermia mask. During the preparation of the hyperthermia mask, the phase change fibers can be filled into the mask surface body preferably.

A method for using the hyperthermia mask of the present invention comprises the following steps: heating the hyperthermia mask by a heating device before use, wherein the heating temperature is higher than the phase change temperature of the phase change materials; a patient with rhinitis wears the hyperthermia mask after heating; since the environmental temperature is lower than the phase change temperature (40° C.-55° C.) of the phase change materials selected by us, the phase change materials in the hyperthermia mask may undergo phase change and release heat to play the role of hyperthermia, wherein the heating device can be a microwave oven or a hair dryer; and certainly, other heating devices can also be used for heating.

The present invention has beneficial effects that the hyperthermia mask of the present invention is based on the phase change heat storage, is convenient to use and has a good hyperthermia effect.

### BRIEF DESCRIPTION OF DRAWINGS

FIG. 1 is a structural schematic diagram of a hyperthermia mask of the present invention.

### REFERENCE NUMERALS

In the FIGURE, 1 refers to a mask surface body; 2 refers to a tensioning belt; 3 refers to phase change fibers; and 4 refers to an opening with a zipper.

### DETAILED DESCRIPTION OF THE INVENTION

In order to better describe the purpose, the technical solution and the advantages of the present invention, the present invention is further described below in combination with drawings and specific embodiments.

### Embodiment 1

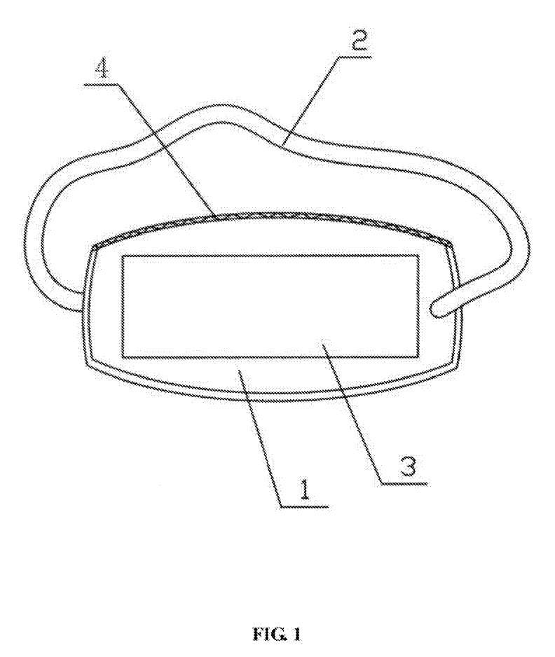

As shown in FIG. 1, a hyperthermia mask of an embodiment of the present invention comprises a mask surface body 1 and a tensioning belt 2. The mask surface body 1 is filled with phase change fibers 3. The phase change fibers 3 are composed of hollow fibers and paraffin filled into the hollow fibers, wherein a melting point of the paraffin is 40° C.-55° C. The paraffin as an organic phase change heat storage material has advantages of high latent heat of phase change, adjustable phase change temperature, stable performance, no poison, etc.

When the phase change fibers 3 are prepared, the paraffin can be filled into the hollow fibers by an adsorption method, a blending method or a dipping method. In the present embodiment, a method for preparing the phase change fibers 3 preferably comprises the following steps: firstly, heating the paraffin to a molten state; then, immersing one end of each hollow fiber in the paraffin, inserting the other end of each hollow fiber into one end of a rubber tube, and sealing a connection port of the hollow fibers and the rubber tube; and finally, connecting the other end of the rubber tube with a vacuum pump to suck the paraffin into the hollow fibers.

In order to prepare the phase change fibers with high latent heat, ensure the hyperthermia time of the hyperthermia mask to be more than 30 minutes and prevent the paraffin from melting and leaking, the filling rate of the paraffin in the hollow fibers is 80%-90% in the present embodiment.

In order to conveniently fill the phase change fibers 3 into the mask surface body 1 and more conveniently replace the phase change fibers 3, an opening 4 with a zipper is further formed in the mask surface body 1; and the phase change fibers 3 are filled into the mask surface body 1 through the opening 4 with the zipper.

In addition, gauzes are arranged on upper surfaces and lower surfaces of the phase change fibers 3 of the present embodiment, so that the phase change fibers can be more conveniently placed in the mask surface body, and the mask has a better heat preservation effect. Certainly, other textiles, such as cotton cloth can also be used to replace the gauzes.

A method for using the hyperthermia mask of the present embodiment comprises the following steps: heating the hyperthermia mask by a heating device before use to melt paraffin; a patient with rhinitis wears the hyperthermia mask after the paraffin is molten; since the environmental temperature is lower than a melting point of the paraffin, when the patient wears the hyperthermia mask, the paraffin is solidified, releases heat and keeps the temperature of the mask close to the phase change temperature of the paraffin, thereby playing a role of hyperthermia.

It should be noted that the hyperthermia mask of the present invention can also be prepared by spinning the phase change fibers. A spinning method is the prior art, wherein the phase change fibers are composed of hollow fibers and the phase change materials filled into the hollow fibers; and the phase change materials have a phase change temperature of 40° C.-55° C.

### Embodiment 2

In order to verify the effect of the hyperthermia mask of the present invention in the treatment of rhinitis, we randomly select 50 patients with rhinitis and inspect relevant symptoms (nasal itching, nasal congestion, nasal mucus and sneezing) of the rhinitis before and after the patients wear the hyperthermia mask of the present invention. The selected patients with rhinitis used the hyperthermia mask of the present invention for hyperthermia twice every day for 30 minutes every time. After one week of hyperthermia, only five patients had mild nasal congestion symptom; after two weeks of hyperthermia, the nasal itching, nasal congestion, nasal mucus and sneezing symptoms of 50 patients were significantly improved, and no patient had nasal itching, nasal congestion, nasal mucus and sneezing situations.

Thus, the hyperthermia mask of the present invention can treat the rhinitis effectively.

Finally, it should be noted that the above embodiments are merely used to illustrate the technical solution of the present invention, not to limit a protection scope of the present invention. Although the present invention is described in detail with reference to the preferred embodiments, those ordinary skilled in the art should understand that the technical solution of the present invention can be modified or equivalently replaced without departing from the substance and the scope of the technical solution of the present invention.

What is claimed is:

1. A hyperthermia mask, comprising a mask surface body and a tensioning belt; wherein the mask surface body is made of phase change fibers or the mask surface body is filled with the phase change fibers; the phase change fibers are composed of hollow fibers and a phase change material filled into the hollow fibers; the phase change material has a phase change temperature of 40° C.-55° C.;
the phase change material is paraffin;
the phase change fibers are prepared by a method of heating the paraffin to a molten state, immersing one end of each hollow fiber in the paraffin, inserting the other end of each hollow fiber into one end of a rubber tube, sealing a connection port of the hollow fibers and the rubber tube and connecting the other end of the rubber tube with a vacuum pump to suck the paraffin into the hollow fibers.

2. The hyperthermia mask of claim 1, wherein a filling rate of the paraffin in the hollow fibers is 80%-90%.

3. The hyperthermia mask of claim 1, wherein an opening with a zipper is further formed in the mask surface body; and the phase change fibers are filled into the mask surface body through the opening with the zipper.

4. The hyperthermia mask of claim 3, wherein textiles are arranged on an upper surface and a lower surface of the phase change fibers.

5. The hyperthermia mask of claim 4, wherein the textiles are gauzes or cotton cloths.
